(19) United States
(12) Patent Application Publication
(10) Pub. No.: US 2011/0190644 A1
(43) Pub. Date: Aug. 4, 2011

(54) Living body pressing device, method of manufacturing same, and blood pressure measuring device

(75) Inventor: Takuro Kohyama, Tokyo (JP)

(73) Assignee: CITIZEN SYSTEMS JAPAN CO., LTD., TOKYO (JP)

(21) Appl. No.: 12/998,272

(22) PCT Filed: Sep. 29, 2009
(86) PCT No.: PCT/JP2009/004996

§ 371 Date: Apr. 4, 2011

(30) Foreign Application Priority Data
Oct. 3, 2008 (JP) 2008-258688

(51) Int. Cl. A61B 5/022; B32B 37/02; B32B 37/14; B32B 38/00

(52) U.S. Cl. 600/499; 156/227

(57) ABSTRACT

Provided are a living body pressing device which can perform effective pressing at both ends in the short-side direction of the device and is reduced in size, a method of manufacturing the living body pressing device, and a blood pressure measuring device. An air bag (2) of a living body pressing device (1) compresses: an air bag body (21) having a single sheet (22) in which both ends thereof parallel to the long-side direction are folded, a fused section (23) where the folded both ends are fused together and which is located at the upper surface of the sheet, and fused sections (24) in the long-side direction where the both ends in the long-side direction are fused respectively; and a reinforcing sheet (3) having a shape corresponding to the upper surface of the air bag body (21) and mounted to the upper surface of the air bag body (21) so as to cover the upper surface.

### TECHNICAL FIELD

The present invention relates to a living body pressing device, a method of manufacturing the same, and a blood pressure measuring device which enables effective pressing at both ends in the arm-axis direction in pressing an arm and also enables a reduction in size.

### BACKGROUND ART

Recently, sphygmomanometers have come into wide use not only in, for example, hospitals but also in general households for the purpose of health care. The sphygmomanometers for the general households are typically automatic sphygmomanometers that are easy to operate.

Automatic sphygmomanometers based on various measurement methods and configurations have been developed, but an oscillometric electronic sphygmomanometer is one of such sphygmomanometers. This oscillometric electronic sphygmomanometer detects a pulse wave superposed on the inner pressure of an air bag, and calculates a blood pressure on the basis of changes in the amplitude of the pulse wave. This oscillometric electronic sphygmomanometer includes a living body pressing device, a pressure sensor, a pump, an exhaust valve, an information processing unit, an operation switch and a display. The living body pressing device (generally called a cuff) is wound around the upper arm, and has an air bag. The pressure sensor, the pump and the exhaust valve are in communication with the air bag via a pipe. The information processing unit is connected to the pressure sensor, the pump and the exhaust valve. The operation switch and the display are connected to the information processing unit.

Living body pressing devices of various configurations are used, the automatic sphygmomanometers.

Now, a conventional living body pressing device is described with reference to the drawings.

(Example of Prior Art)

FIG. 8 is a schematic diagram of a living body pressing device according to a conventional example, wherein (a) shows an extended view of its front side, and (b) shows an extended view of its rear side.

In FIG. 8, a living body pressing device 100 comprises a belt-shaped member 110 formed by holding an air bag 120 between an external cover 111 and an internal cover 112, a ring-shaped clip 13 attached to one end of the belt-shaped member 110 in the long-side direction, and a hook-and-loop fastener 130 which is inserted into the clip 13 and which fastens the other end of the belt-shaped member 110 folded back via the clip 13.

FIG. 9 is a schematic diagram of essential parts of the air bag of the living body pressing device according to the conventional example, wherein (a) shows a plan view, and (b) shows a C-C enlarged sectional view for illustrating a pressing condition. In FIG. 9(b), a joint 25 and the internal cover 112 are not shown for clarity.

In FIG. 9, the air bag 120 has, for example, an upper side sheet 121, a lower side sheet 122, and a fused section 123 where the peripheral edges of these sheets are fused together. This air bag 120 has a simple structure and can be easily manufactured, and manufacturing costs can therefore be reduced.

When the living body pressing device 100 is wound around an arm and air is supplied to the air bag 120 from the joint 25, the air bag 120 inflates as shown in FIG. 9(b), and presses the arm.

The upper side sheet 121 and the lower side sheet 122 of this air bag 120 are generally made of the same material. Therefore, the upper side sheet 121 and the lower side sheet 122 have the same thickness. Moreover, the air bag 120 is a fluid bag which inflates or deflates as a fluid (generally air) comes in or out.

Furthermore, various techniques have been developed to prevent air from easily leaking from the fused section of the air bag or to enable effective pressing at both ends in the arm-axis direction.

For example, Patent document 1 discloses a technique of a method of manufacturing a sphygmomanometer cuff air bag. This sphygmomanometer cuff air bag is characterized in that opposite two sides at both ends of a single square resin sheet are superposed on each other inside and outside so that the outer surface of one end is in contact with the inner surface of the other end, and the superposed portion is fused by, for example, a high-frequency welder into a cylindrical shape, and the cylinder of the resin sheet is made flat to position the fused portion substantially in the center of one surface, such that two open sides at both ends of the flat cylindrical part are fused and thus closed, respectively.

According to this technique, the sphygmomanometer cuff air bag can be manufactured so that air does not easily leaks out.

Furthermore, Patent document 2 discloses a technique of a sphygmomanometer cuff comprising a fluid bag (air bag) which inflates or deflates as a fluid comes in or out. The fluid bag of this sphygmomanometer cuff has an outer wall located outside, an inner wall located inside, sidewalls which are respectively connected to the both ends of the outer wall and the inner wall in the winding direction and which are folded in the inward direction of the fluid bag, and a coupler which connects the these sidewalls in the fluid bag.

According to this technique, the fluid bag does not change in width even when inflated and does not inflate in the width direction, and can thus maintain the original shape when both inflated and deflated.

Furthermore, Patent document 3 discloses a technique of a sphygmomanometer cuff comprising a fluid bag (air bag) which inflates or deflates as a fluid comes in or out. The fluid bag of this sphygmomanometer cuff has a first fluid chamber which is substantially rectangular when viewed in plan and which is located outside, and a second fluid chamber which is substantially rectangular when viewed in plan and which is located inside. Moreover, the fluid bag has a junction where the opposite surfaces of the first and second fluid chambers are joined together in regional parts smaller than their regions viewed in plan. The fluid bag also has a hole through which the first and second fluid chambers are connected into the region surrounded by the junction.

According to this technique, the fluid bag does not change in width even when inflated and does not inflate in the width direction when inflated, and can thus maintain the original shape when both inflated and deflated.

Furthermore, Patent document 4 discloses a technique of a sphygmomanometer cuff comprising therein a flexible bending plate and an air bag. This sphygmomanometer cuff is characterized in that the size of the bending plate in an expanded shape is smaller than the size of the air bag in an expanded shape.

According to this technique, the size of the bending plate attached to the cuff is smaller than the size of the air bag, so that a subject can feel more comfortable with the wound cuff without pain.

Furthermore, Patent document 5 discloses a technique of a blood pressure measurement cuff belt comprising a flat rubber bag and a fabric belt provided so as to cover the rubber bag. An expansion preventing member is provided on the side surface of the rubber bag that does not face a subject to prevent the rubber bag from being unnecessarily expanded by pressure applied by the rubber bag.
Patent document 1: Japanese Patent Publication Laid-open No. 2003-52651
Patent document 2: Japanese Patent Publication Laid-open No. 2001-224558
Patent document 3: Japanese Patent Publication Laid-open No. 2003-325462
Patent document 4: Japanese Patent Publication Laid-open No. 2002-238864
Patent document 5: Japanese Patent Publication Laid-open No. 53-10594

### DISCLOSURE OF THE INVENTION

However, in the living body pressing device 100 described above, the fused section 123 does not at all contribute to the pressing of a living body (properly abbreviated as an arm) at both ends in the arm-axis direction as shown in FIG. 9(b). Moreover, both ends of the lower side sheet 122 connected to the fused section 123 are slanted, and these portions do not press the arm. That is, when air is supplied to the air bag 120, the length of the air bag 120 in the arm-axis direction is L₀+2×ΔL₀, but the effective pressing length in the arm-axis direction is L₀, so that the parts at both ends corresponding to the length 2×ΔL₀ do not press the arm. Here, in order to accurately measure the blood pressure, a predetermined effective pressing length in the arm-axis direction has to be ensured. Thus, the problem of the living body pressing device 100 is that if the predetermined effective pressing length in the arm-axis direction is to be ensured, a dimension W₀ of the belt-shaped member 110 in the arm-axis direction shown in FIG. 8 is increased, and the living body pressing device 100 cannot be reduced in size.

Furthermore, there is an improved technique whereby the upper side sheet 121 and the lower side sheet 122 are varied in thickness to reduce the slanted portions at the both ends of the lower side sheet 122 in the living body pressing device 100 (e.g., an improved technique whereby the upper side sheet 121 is made thicker than the lower side sheet 122). However, a problem of this technique is that the sheets different in thickness have to be managed and that the man-hour of management is increased. Another problem of this technique is that the portion of the fused section 123 does not at all contribute to the pressing of the arm so that the living body pressing device 100 cannot be well reduced in size.

According to the above-described technique of Patent document 1, although not shown, the upper surface and lower surface of the air bag and its surfaces (side surfaces) located at the both ends in the arm-axis direction are substantially equally easy to inflate and curve. When wound around the arm, this air bag is held between the external cover and the arm, and the centers of its upper surface and lower surface tend to inflate. Moreover, when the cuff (living body pressing device) is loosely wound around the arm, the amount of parts protruding in the arm-axis direction that do not contribute to the pressing of the arm is increased at the both ends in the arm-axis direction. That is, the problem is that the effective pressing length in the arm-axis direction is reduced and that effective pressing cannot be obtained at the both ends in the arm-axis direction.

Still another problem of the above-described techniques of Patent documents 2 and 3 is that effective pressing can be obtained at the ends in the arm-axis direction but the complex structures prevent the reduction of the manufacturing costs.

Moreover, the above-described technique of Patent document 4 is intended to provide the flexible bending plate (sheet core) in order for a subject to feel more comfortable with the wound cuff without pain, and cannot solve the above-mentioned problems.

Further, according the above-described technique of Patent document 5, the expansion preventing plate is disposed outside the rubber bag to concentrate pressure on the body surface. However, since the rubber bag inflates in a circular-arc shape, the expansion preventing plate and the rubber bag are separated at the end of the expansion preventing plate in the arm-axis direction. Thus, the problem is that this technique is not capable of holding in the direction of the body surface and does not enable effective pressing at the end in the arm-axis direction.

The present invention has been suggested to solve the problems of the conventional techniques described above, and is directed to provide a living body pressing device, a method of manufacturing the same, and a blood pressure measuring device which enables effective pressing at both ends in the short-side direction and also enables a reduction in size.

In order to achieve the foregoing object, a living body pressing device according to the present invention comprises a fluid bag which inflates or deflates as a fluid comes in or out and which is wound around a living body, wherein a reinforcing sheet is provided on a side of the fluid bag that does not face the living body.

Consequently, the inflation amount of the upper surface of the fluid bag is suppressed, and the inflation amounts of the lower surface and side surfaces of the fluid bag are increased. Thus, the lower surface of the fluid bag can more uniformly press the living body.

Moreover, the living body pressing device has a simple structure, and manufacturing costs can therefore be reduced.

Preferably, the fluid bag is rectangular, and the both ends of the reinforcing sheet on the long sides of the fluid bag are secured.

Consequently, when the fluid (generally, air) is supplied to the fluid bag, the protrusion amount in the short-side direction that does not contribute to the pressing of the living body is reduced in the curved side surface of the fluid bag in the short-side direction. That is, the effective pressing length in the short-side direction is increased, so that effective pressing can be obtained at the end in the short-side direction, and the living body pressing device can be reduced in size.

Preferably, the reinforcing sheet is secured in a region smaller than the fluid bag.

This ensures that the inflation amount of the side surface of the fluid bag can be increased by the fluid bag outside the reinforcing sheet.

Preferably, the fluid bag is formed by folding a single sheet.

Consequently, the living body pressing device can have a simple structure, and manufacturing costs can therefore be reduced.

Preferably, one of fused sections in a short-side direction, where both ends of the reinforcing sheet in the short-side direction and the upper surface of the fluid bag are fused, overlaps the fused section of the upper surface.

Consequently, one of the fused sections in the short-side direction and the fused section of the upper surface can be fused at the same time. Thus, operating efficiency can be improved.

Preferably, a sheet core is interposed between the upper surface of the fluid bag and the reinforcing sheet.

This ensures that the inflation amount of the upper surface of an air bag body can be suppressed.

Furthermore, according to the present invention, a method of manufacturing a living body pressing device which is wound around a living body and which comprises a fluid bag that inflates or deflates as a fluid comes in or out and that is equipped with a reinforcing sheet, and a cover including the fluid bag therein. The method comprises the steps of: to manufacture the fluid bag, folding both ends of a single sheet parallel to a long-side direction; fusing the folded both ends and at least one of the both ends of the reinforcing sheet in the short-side direction at the same time; and fusing the both ends in the long-side direction, respectively.

Consequently, operating efficiency can be improved, and a manufacturing facility can be shared. Thus, manufacturing costs can be reduced.

A blood pressure measuring device according to the present invention is configured to use the living body pressing device of one of claims 1 to 6.

Thus, the present invention is also effective as the blood pressure measuring device, so that it is possible to provide a blood pressure measuring device which enables effective pressing at both ends in the short-side direction in pressing a living body and also enables a reduction in size.

According to the living body pressing device, the method of manufacturing the same, and the blood pressure measuring device, effective pressing can be obtained at both ends in the short-side direction, and the living body pressing device can be reduced in size.

### BRIEF DESCRIPTION OF THE DRAWINGS

FIG. 1 is a schematic diagram of a living body pressing device according to a first embodiment of the present invention, wherein (a) shows an extended view of its front side and (b) shows an extended view of its rear side;

FIG. 2 is a schematic diagram of an air bag of the living body pressing device according to the first embodiment of the present invention, wherein (a) shows a plan view, and (b) shows an A-A enlarged sectional view;

FIG. 3 is a schematic diagram for illustrating a pressing condition of the air bag of the living body pressing device according to the first embodiment of the present invention, wherein (a) shows an A-A enlarged sectional view, and (b) shows an enlarged sectional view of a comparative example;

FIG. 4 is a schematic diagram of an air bag of a living body pressing device according to a second embodiment of the present invention, wherein (a) shows a plan view, and (b) shows a B-B enlarged sectional view;

FIG. 5 shows a schematic flowchart for illustrating a method of manufacturing the living body pressing device according to one embodiment of the present invention;

FIG. 6 is a schematic diagram for illustrating the method of manufacturing the living body pressing device according to one embodiment of the present invention, wherein (a) shows a plan view for illustrating a condition in which one end of a reinforcing sheet is fused, and (b) shows an enlarged sectional view for illustrating collective fusing;

FIG. 7 shows a schematic block diagram of essential parts of an oscillometric electronic sphygmomanometer according to one embodiment of the present invention;

FIG. 8 is a schematic diagram of a living body pressing device according to a conventional example, wherein (a) shows an extended view of its front side, and (b) shows an extended view of its rear side; and

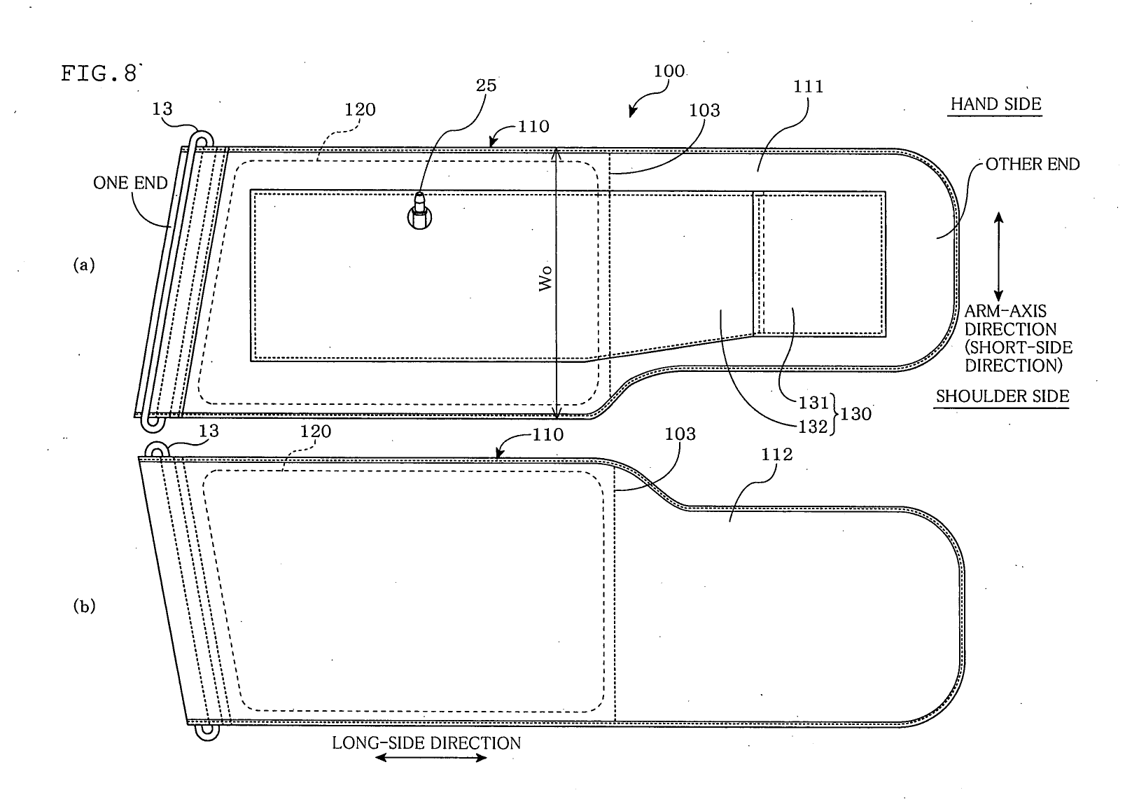

FIG. 9 is a schematic diagram of essential parts of the air bag of the living body pressing device according to the conventional example, wherein (a) shows a plan view, and (b) shows a C-C enlarged sectional view for illustrating a pressing condition.

### BEST MODE FOR CARRYING OUT THE INVENTION

### First Embodiment of Living Body Pressing Device

A first embodiment of a living body pressing device of the present invention will be described below with reference to the drawings.

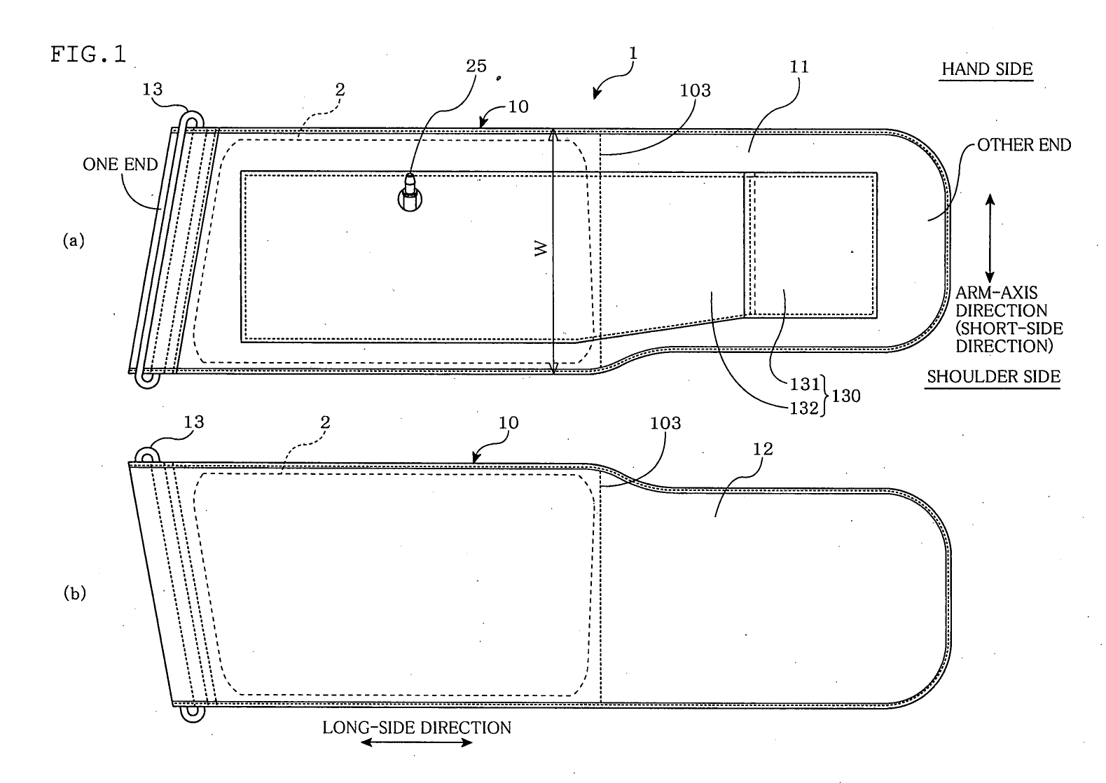

FIG. 1 is a schematic diagram of the living body pressing device according to the first embodiment of the present invention, wherein (a) shows an extended view of its front side and (b) shows an extended view of its rear side.

In FIG. 1, a living body pressing device 1 is used as an automatic sphygmomanometer cuff, and is wound around an upper arm of a user.

This living body pressing device 1 comprises a belt-shaped member 10 including an air bag 2 as a fluid bag, a clip 13, and a hook-and-loop fastener 130.

(Air Bag)

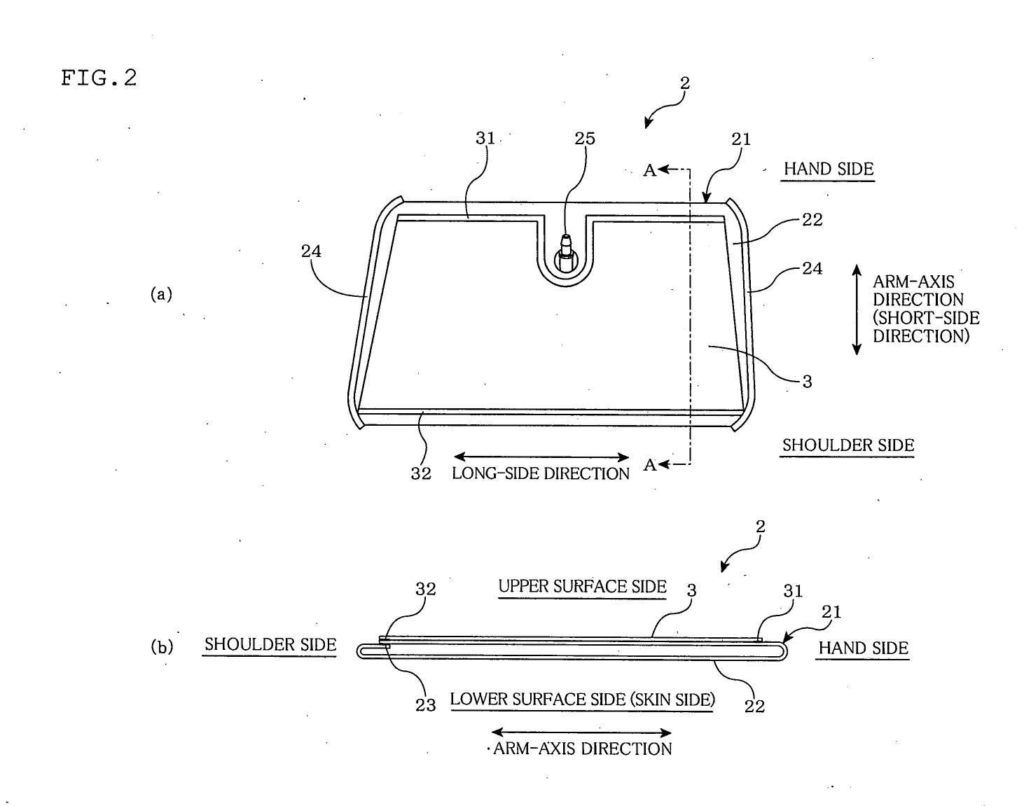

FIG. 2 is a schematic diagram of the air bag of the living body pressing device according to the first embodiment of the present invention, wherein (a) shows a plan view, and (b) shows an A-A enlarged sectional view. For the structure of the air bag 2 to be easily understood, FIG. 2(b) shows a sectional view in which a joint 25 is omitted and the air bag is flat with substantially no air supplied thereto.

In FIG. 2, the air bag 2 is rectangular, and comprises an air bag body 21, a reinforcing sheet 3, and the joint 25.

The air bag body 21 is manufactured from a single resin flat sheet 22. Both ends of this sheet 22 parallel to the long-side direction are folded, and these folded both ends are fused in the upper surface, and the both ends in the long-side direction are fused. That is, the air bag body 21 has the single sheet 22 in which both ends thereof parallel to the long-side direction are folded, a fused upper surface section 23 where the folded both ends of the sheet 22 are fused together and which is located at the upper surface of the sheet 22, and fused sections 24 in the long-side direction, where the both ends in the long-side direction are fused respectively.

Furthermore, the width dimension of the fused section, for example, the fused upper surface section 23 and the fused sections 24 in the long-side direction is generally, but not limited to, 1 to 5 mm.

The sheet 22 is generally produced easily from a film-like sheet material by punching. Moreover, this sheet material is flexible, and is also elastic or stretchable.

In addition, the joint 25 is generally attached to the sheet 22 before the fused upper surface section 23 is processed.

The reinforcing sheet 3 is equal in material and thickness to the sheet 22. Thus, there is no need to manage the sheets different in material or thickness, so that the man-hour of management can be reduced. Moreover, the reinforcing sheet 3 is provided on the side of the air bag 2 that does not face a living body.

This reinforcing sheet 3 has a rectangular shape substantially corresponding to the upper surface of the air bag body 21. That is, the reinforcing sheet 3 has about the same shape as the outer shape of the upper surface of the air bag body 21 that is reduced several mm inward, and the reinforcing sheet 3 is secured in a region smaller than the air bag body 21. This ensures that the inflation amount of the side surface of the air bag body 21 can be increased by the air bag body 21 outside the reinforcing sheet 3.

Moreover, the reinforcing sheet 3 has a U-shaped cut-out formed at the position corresponding to the joint 25.

The both ends of the reinforcing sheet 3 according to the present embodiment on the long sides of the air bag 2 (the both ends in the short-side direction (arm-axis direction)) are fused to the upper surface of the air bag body 21. That is, the reinforcing sheet 3 is mounted to the upper surface of the air bag body 21 so as to cover this upper surface by a hand-side fused section 31 which has substantially straight two lines and a U shape and by a shoulder-side fused section 32 which has a shape of substantially straight one line.

As a result, the upper surface (the single reinforcing sheet 3 and the single sheet 22) of the air bag 2 located between the hand-side fused section 31 and the shoulder-side fused section 32 is more difficult to stretch than the lower surface (the single sheet 22) of the air bag 2. Moreover, the upper surface (the single reinforcing sheet 3 and the single sheet 22) of the air bag 2 located between the hand-side fused section 31 and the shoulder-side fused section 32 is more difficult to curve than the lower surface (the single sheet 22) of the air bag 2.

Here, the shoulder-side fused section 32, where the shoulder-side end of the reinforcing sheet 3 in the arm-axis direction is fused to the upper surface of the air bag body 21, preferably overlap the fused upper surface section 23 of the air bag body 21. Consequently, the shoulder-side fused section 32 and the fused upper surface section 23 can be fused at the same time. Thus, operating efficiency can be improved.

Furthermore, the air bag 2 has, in its upper surface, the protruding joint 25 to be coupled to the pipe of an oscillometric electronic sphygmomanometer. The joint 25 is exposed through an external cover 11 and a loop surface 132.

Although a double-faced tape is generally used to fix the air bag 2, the air bag 2 may not be exclusively fixed by the double-faced tape but may be fixed by adhesive bonding or stitching.

(Belt-Shaped Member)

The belt-shaped member 10 is configured to hold the air bag 2 between the external cover 11 and an internal cover 12 as shown in FIG. 1.

Substantially rectangular resin fabrics are generally used for the external cover 11 and the internal cover 12. The internal cover 12 is made of a thin, soft and stretchable fabric to provide a comfortable touch when in contact with the skin. In contrast with the internal cover 12, the external cover 11 is made of a thick, strong and unstretchable fabric.

The peripheral edges of the external cover 11 and the internal cover 12 are stitched together by a thread via an edge cover so that the air bag 2 is held therebetween. The external cover 11 and the internal cover 12 are also stitched together by a thread 103 substantially in the center of the long-side direction along the arm direction.

Furthermore, the width dimension of a portion of the external cover 11 and the internal cover 12, which is located between the thread 13 and the other end, is smaller than that of a portion of the external cover 11 and the internal cover 12, which is located between the thread 13 and the one end. Thus, the external cover 11 and the internal cover 12 easily adapt to the arm shape.

(Clip)

The clip 13 is a ring-shaped member made of a round bar, and the other end of the belt-shaped member 10 is inserted into the clip 13. This clip 13 is attached to one end of the belt-shaped member 10 in the long-side direction.

This clip 13 is diagonally attached at an angle of about 80° with the lower side (shoulder-side side) of the belt-shaped member 10. Thus, the living body pressing device 1 can be wound around even a muscular upper arm substantially in close contact state.

(Hook-and-Loop Fastener)

According to the present embodiment, the hook-and-loop fastener 130 is provided as a fastener which is inserted in the clip 13 and which fastens the other end of the belt-shaped member 10 folded back via the clip 13.

The hook-and-loop fastener 130 comprises a hook surface 131 and the loop surface 132. The hook surface 131 is substantially quadrate, and is stitched to the surface of the other end of the external cover 11 by a thread. The loop surface 132 is substantially rectangular, and is stitched to the surface of the external cover 11 by a thread from the hook surface 131 to the vicinity of one end thereof.

Consequently, the living body pressing device 1 can be easily attached and removed. For example, when the living body pressing device 1 is attached to the left upper arm, the living body pressing device 1 can be attached and removed by pinching the other end with the right hand.

Although the hook-and-loop fastener 130 is used as a fastener according to the present embodiment, the hook-and-loop fastener 130 is not exclusively used.

Now, the operation of the living body pressing device 1 having the configuration described above is described.

First, the user to measure the blood pressure puts the other end of the belt-shaped member 10 through the clip 13, and inserts the arm into the expanded living body pressing device 1. When the living body pressing device 1 reaches the upper arm, the user pulls the other end. As a result, the belt-shaped member 10 is wound around the upper arm and folded back via the clip 13, and the hook surface 131 is joined to the loop surface 132. At the same time, the living body pressing device 1 is wound around the arm so that the external cover 11 is not too loose and so that the air bag 2 does not extremely press the arm.

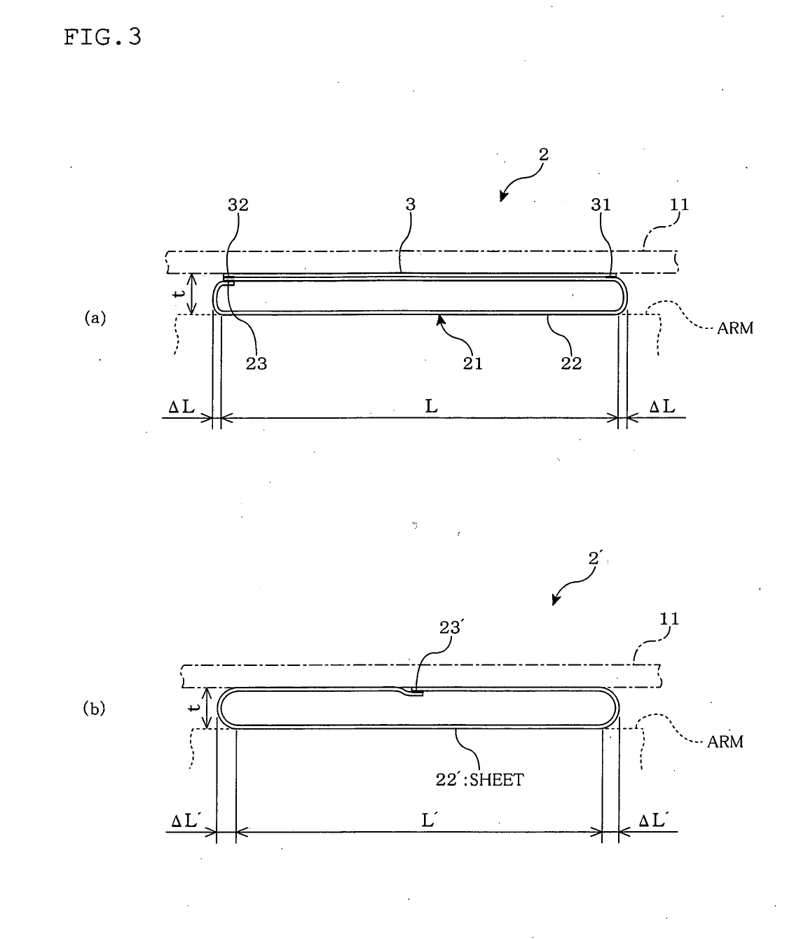

FIG. 3 is a schematic diagram for illustrating a pressing condition of the air bag of the living body pressing device according to the first embodiment of the present invention, wherein (a) shows an A-A enlarged sectional view, and (b) shows an enlarged sectional view of a comparative example. In FIG. 3, the joint 25 and the internal cover 12 are not shown for clarity.

In FIG. 3(a), the living body pressing device 1 is wound around the arm, and air is supplied to the air bag 2 by an air supply unit such as an air supply balloon (not shown). The air bag 2 is inflated by the supplied air, and the external cover 11 is tightened, and then the arm is pressed.

Here, the external cover 11 is a thick, strong and unstretchable fabric. When wound around the arm, the external cover 11 is curved in the long-side direction, and the arm is also slightly curved in accordance with the muscles. That is, the external cover 11, the air bag 2 and the arm are not ideal elastic bodies or rigid bodies, and are flexible or slightly elastic. Moreover, the living body pressing device 1 is wound around the arm in various conditions. Therefore, in FIG. 3, these components are linearly represented for clarity.

As described above, the upper surface (the single reinforcing sheet 3 and the single sheet 22) of the air bag 2 located between the hand-side fused section 31 and the shoulder-side fused section 32 is more difficult to stretch than the lower surface (the single sheet 22) of the air bag 2. Moreover, the upper surface (the single reinforcing sheet 3 and the single sheet 22) of the air bag 2 located between the hand-side fused section 31 and the shoulder-side fused section 32 is more difficult to curve than the lower surface (the single sheet 22) of the air bag 2. Thus, the inflation amount of the upper surface of the air bag body 21 is suppressed, and the inflation amounts of the lower surface and side surface of the air bag body 21 are relatively increased. That is, when air is supplied to the air bag 2, the air bag 2 first inflates in the center of the lower surface, and this inflation moves to the both ends in the arm-axis direction in conformity to the shape of the arm. As a result, the lower surface of the air bag body 21 can more uniformly press the arm.

Furthermore, elasticity and pliability sharply change at the boundary between the hand-side fused section 31 and the side surface of the air bag body 21 and at the boundary between the fused upper surface section 23 as well as the shoulder-side fused section 32 and the side surface of the air bag body 21. Therefore, when air is supplied, the living body pressing device 1 is deformed into a discontinuous shape at these boundaries. In contrast, an air bag 2′ shown in FIG. 3(b), for example, has a sheet 22′ the both ends of which are folded, and a fused upper surface section 23′ located substantially in the center of the upper surface. Thus, the air bag 2′ has about the same elasticity and pliability as a whole. When air is supplied to the air bag 2′, its side surface curves in a substantially semicircular shape.

That is, when air is supplied to the air bag 2, the lower surface of the air bag body 21 first inflates in conformity to the shape of the arm as described above. When this inflation reaches the ends in the arm-axis direction, the living body pressing device 1 is deformed into a discontinuous shape at the above-mentioned boundaries. Therefore, an effective pressing length (L>L′) in the arm-axis direction is increased, and a protrusion amount ΔL (ΔL<ΔL') in the arm-axis direction that does not contribute to the pressing of the arm is reduced in the curved side surface in the arm-axis direction.

Therefore, even if the living body pressing device 1 is loosely wound around the arm (when a space t is great), effective pressing can be obtained at the ends in the arm-axis direction when air is supplied to the air bag 2.

Furthermore, as described above, when air is supplied to the air bag 2, the part of the upper surface closer to the hand side than the hand-side fused section 31 and the part of the upper surface closer to the shoulder side than the shoulder-side fused section 32 in FIG. 2(b) are curved and function as side surfaces as shown in FIG. 3(a). Thus, if the part of the upper surface closer to the hand side than the hand-side fused section 31 and the part of the upper surface closer to the shoulder side than the shoulder-side fused section 32 are increased in width dimension, the living body pressing device 1 can also adapt to a greater space t.

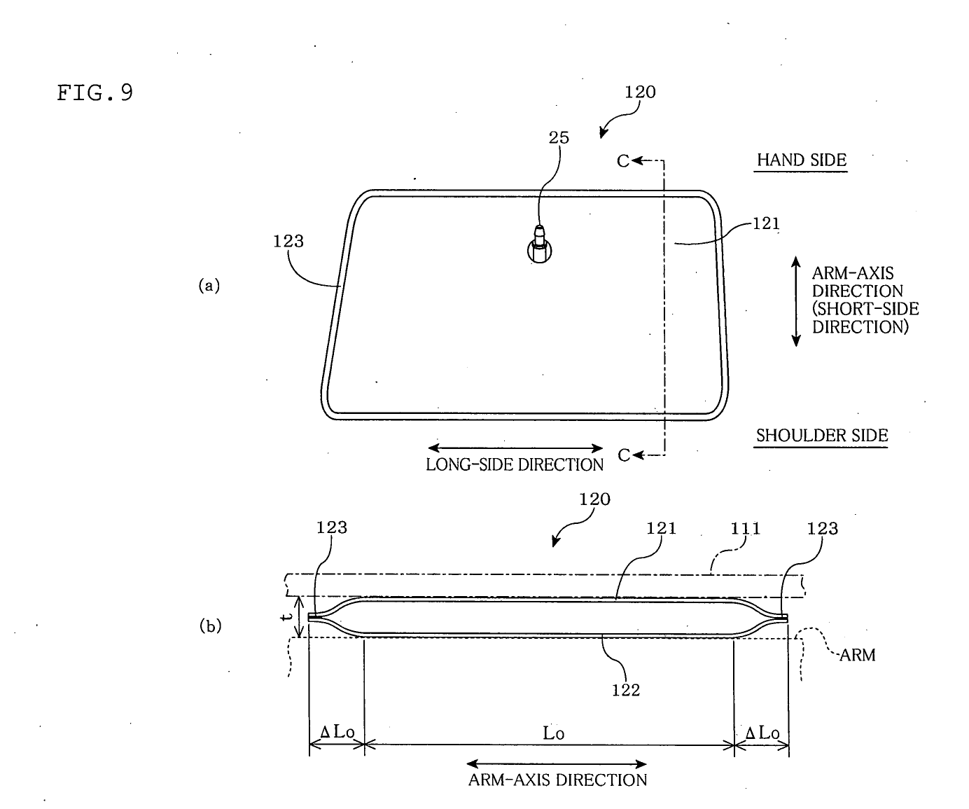

The protrusion amount ΔL of the living body pressing device 1 in the arm-axis direction is much smaller than ΔL₀ shown in FIG. 9(b), therefore, a width dimension W (W<W₀) in FIG. 1 can be reduced, and the living body pressing device 1 can be reduced in size.

Moreover, since the air bag 2 of the living body pressing device 1 has a simple structure, manufacturing costs can therefore be reduced.

As described above, according to the living body pressing device 1 of the present embodiment, in the air bag 2, the effective pressing length (L>L') in the arm-axis direction is increased, and the protrusion amount ΔL (ΔL<ΔL') in the arm-axis direction that does not contribute to the pressing of the arm is reduced in the curved side surface in the arm-axis direction. That is, the living body pressing device 1 enables effective pressing at both ends in the arm-axis direction and also enables a reduction in size.

### Second Embodiment of Living Body Pressing Device

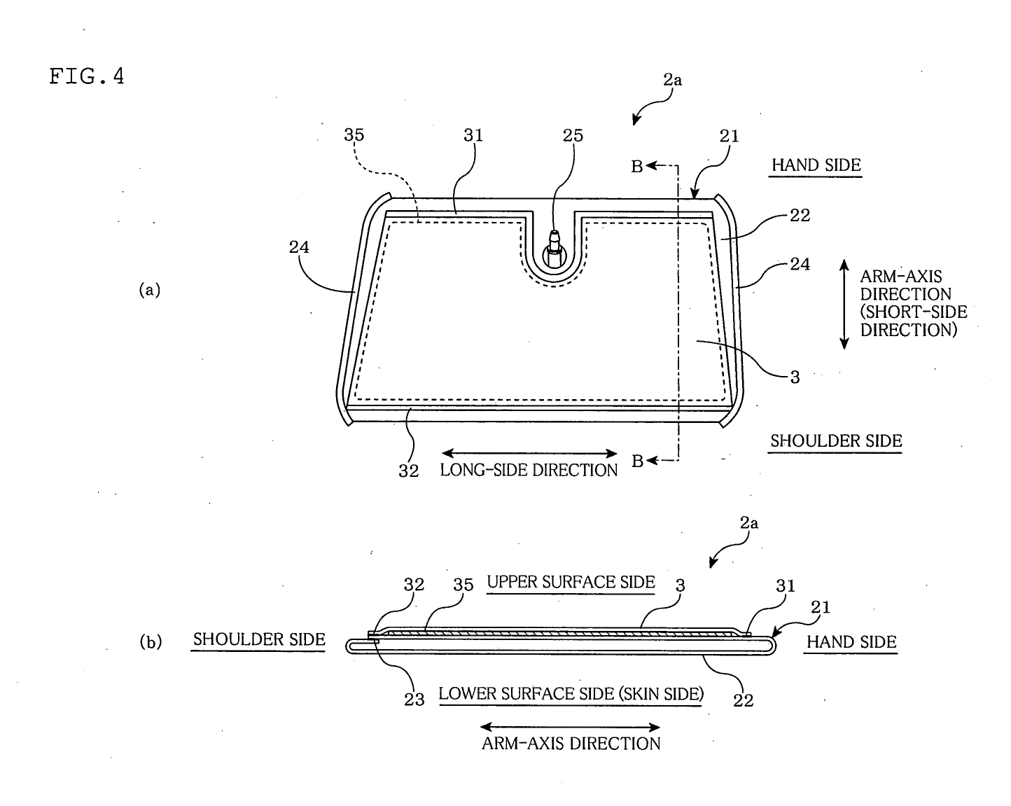

FIG. 4 is a schematic diagram of an air bag of a living body pressing device according to a second embodiment of the present invention, wherein (a) shows a plan view, and (b) shows a B-B enlarged sectional view. For the structure of an air bag 2a to be easily understood, FIG. 4(b) shows a sectional view in which a joint 25 is omitted and the air bag is flat with substantially no air supplied thereto.

In FIG. 4, the living body pressing device according to the present embodiment is different from the living body pressing device 1 according to the first embodiment in that the air bag 2a includes a sheet core 35 between the upper surface of an air bag body 21 and a reinforcing sheet 3. In other respects, the living body pressing device according to the present embodiment is substantially similar in configuration to the living body pressing device 1.

Therefore, in FIG. 4, components similar to those in FIG. 2 are provided with the same reference signs and are not described in detail.

The sheet core 35 is a flexible resin thin plate. This sheet core 35 is generally produced easily from a sheet material by punching. Moreover, this sheet material is flexible, and bends in conformity to the shape of the arm. This sheet material is also resilient to restore its original shape when bent, and, for example, is not easily broken.

Moreover, the sheet core 35 has a shape substantially corresponding to the reinforcing sheet 3. That is, the sheet core 35 has about the same shape as the outer shape of the reinforcing sheet 3 that is reduced several mm inward. Thus, the sheet core 35 is interposed between the upper surface of the air bag body 21 and the reinforcing sheet 3 and thereby fixed. In addition, the sheet core 35 is not exclusively fixed in this manner, and may be fixed by adhesive bonding or by a double-faced tape.

The upper surface (the single reinforcing sheet 3, the sheet core 35, and a single sheet 22) of the air bag 2a located between a hand-side fused section 31 and a shoulder-side fused section 32 is much more difficult to deform than the lower surface (the single sheet 22) of the air bag 2a. That is, when the living body pressing device according to the present embodiment is wound around the arm, the air bag 2a curves in the long-side direction in accordance with the arm shape, but is substantially linear in the arm-axis direction. Therefore, the upper surface of the air bag body 21 does not substantially inflate, and the inflation amounts of the lower surface and side surface of the air bag body 21 are relatively increased. As a result, the lower surface of the air bag body 21 can more uniformly press the arm than in the first embodiment.

Furthermore, substantially as in the first embodiment, elasticity and pliability sharply change at the boundary between the hand-side fused section 31 and the side surface of the air bag body 21 and at the boundary between a fused upper surface section 23 as well as the shoulder-side fused section 32 and the side surface of the air bag body 21. Moreover, according to the present embodiment, the part closer to the central side than the hand-side fused section 31 and the part closer to the central side than the fused upper surface section 23 and the shoulder-side fused section 32 are significantly difficult to deform owing to the sheet core 35. As a result, the hand-side fused section 31 as well as the fused upper surface section 23 and the shoulder-side fused section 32 are also difficult to deform.

Consequently, although not shown, the air bag 2a has a further increased effective pressing length in the arm-axis direction than in the first embodiment, and the protrusion amount in the arm-axis direction that does not contribute to the pressing of the arm is further reduced in the curved side surface in the arm-axis direction. Moreover, even when the living body pressing device according to the present embodiment is wound around the arm in various conditions, the above-described advantages can be more reliably obtained.

As described above, according to the living body pressing device of the present embodiment, the arm can be more uniformly pressed and effective pressing can be more reliably obtained at both ends in the arm-axis direction than in the first embodiment.

### One Embodiment of Living Body Pressing Device Manufacturing Method

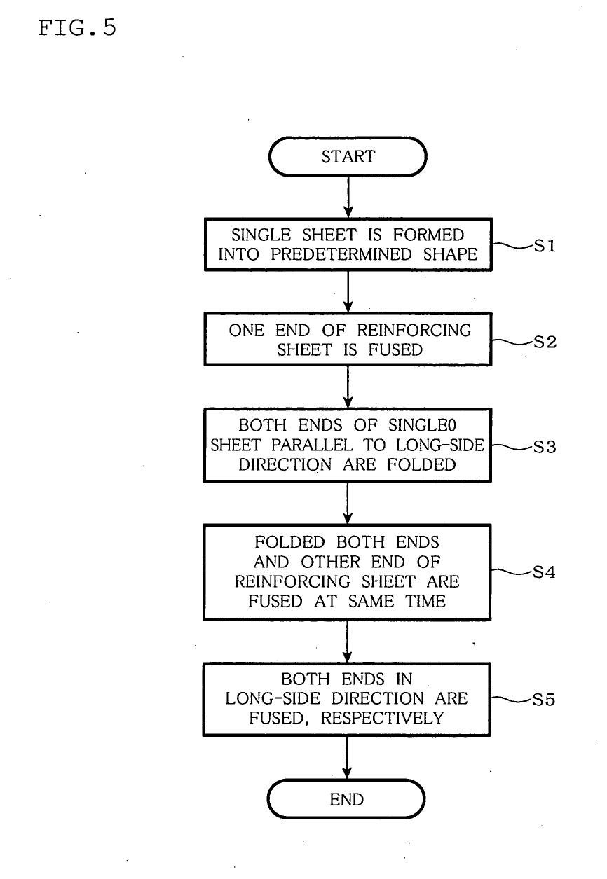

FIG. 5 shows a schematic flowchart for illustrating a method of manufacturing the living body pressing device according to one embodiment of the present invention.

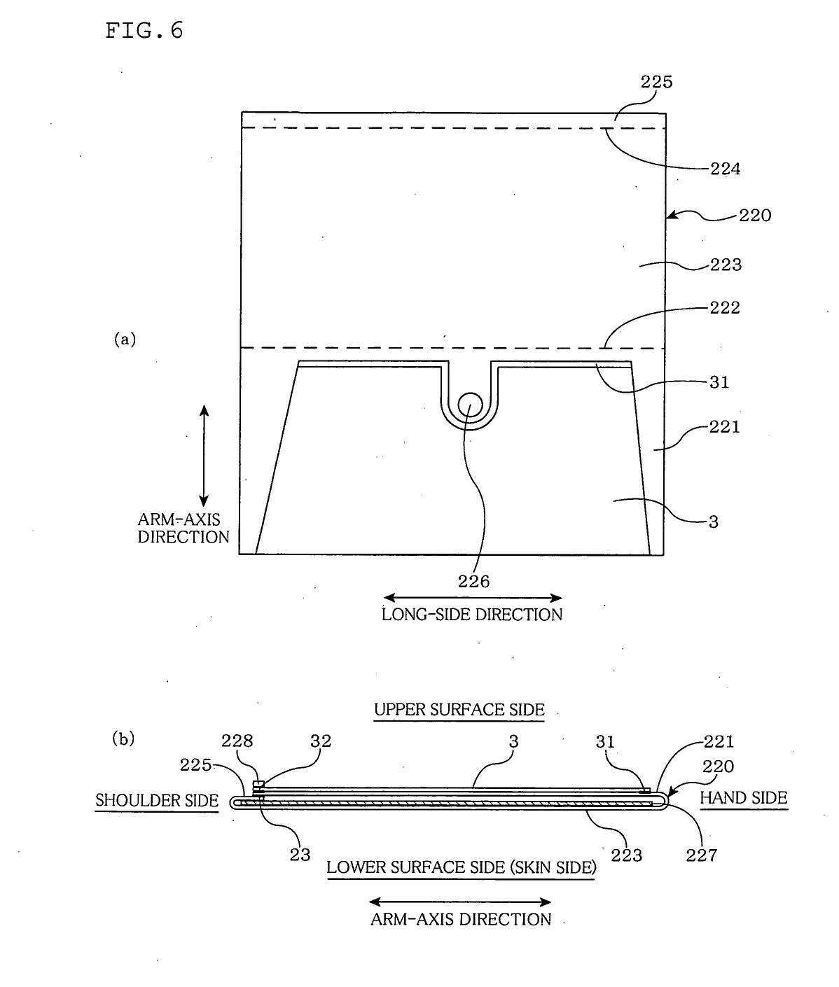

FIG. 6 is a schematic diagram for illustrating the method of manufacturing the living body pressing device according to one embodiment of the present invention, wherein (a) shows a plan view for illustrating a condition in which one end of a reinforcing sheet is fused, and (b) shows an enlarged sectional view for illustrating collective fusing.

In FIGS. 5 and 6, the living body pressing device manufacturing method according to the present embodiment is a method of manufacturing the living body pressing device 1 according to the first embodiment described above.

This living body pressing device manufacturing method has the following steps to manufacture an air bag 2:

First, a single sheet 220 is formed into a predetermined shape by, for example, punching (step 1).

As shown in FIG. 6(a), the sheet 220 is substantially rectangular, and has an upper surface portion 221, a lower surface portion 223 and an upper surface portion 225 in the arm-axis direction. A joint hole 226 is formed in the upper surface portion 221.

Here, the shapes of the both ends of the sheet 220 in the long-side direction do not correspond to the air bag 2. Therefore, this part is formed by use of, for example, a pig after fusing or substantially simultaneously with the fusing in the later-described step (step 5) of the fused sections 24 in the long-side direction. The procedure of forming the both ends of the sheet 220 in the long-side direction are not limited to this, and, for example, the both ends thereof may be formed into shapes corresponding to the air bag 2 in step S1.

A reinforcing sheet 3 formed into a predetermined shape is then fused to a predetermined position (step S2).

A hand-side fused section 31 is formed by this fusing. Although first fusing is performed for the hand-side fused section 31 according to the present embodiment, the present invention is not limited to this. For example, the hand-side fused section 31′ may be simultaneously fused by use of two hot bars when a fused upper surface section 23 and a shoulder-side fused section 32 that will be described later are fused. As a result, the number of times of the fusing can be reduced.

The both ends of the single sheet 220 parallel to the long-side direction are then folded (step S3).

That is, the sheet 220 is turned upside down. Further, the upper surface portion 221 is folded from a folding position 222, and the upper surface portion 225 is folded from a folding position 224. At the same time, as shown in FIG. 6(b), the end of the upper surface portion 221 is placed on the end of the upper surface portion 225, and the end (the end facing the hand-side fused section 31) of the reinforcing sheet 3 is placed on the end of the upper surface portion 221.

The folded both ends and the other end of the reinforcing sheet 3 in the arm-axis direction are then fused at the same time (step S4).

That is, a heat-resistant plate 227 is placed on the lower surface portion 223. Further, a hot bar 228 is pressed down, such that the end of the upper surface portion 225 and the end of the upper surface portion 221 are fused, and the end of the upper surface portion 221 and the end of the reinforcing sheet 3 are fused (collectively fused). The fused upper surface section 23 and the shoulder-side fused section 32 are formed by this fusing. In the present embodiment, this fusing is second fusing.

The heat-resistant plate 227 is then removed.

The both ends in the long-side direction are then fused, respectively (step S5).

As shown in FIG. 2(a), a pair of fused sections 24 in the long-side direction are formed by this third fusing. Moreover, in the present embodiment, the outer portions of the fused sections 24 in the long-side direction are cut by use of, for example, a pig after fusing or substantially simultaneously with the fusing.

As described above, according to the living body pressing device manufacturing method of the present embodiment, the fused upper surface section 23 and the shoulder-side fused section 32 can be fused in one operation in step S4. That is, operating efficiency can be improved, and a manufacturing facility can be shared. Thus, manufacturing costs can be reduced.

### One Embodiment of Blood Pressure Measuring Device

The present invention is also effective as an invention of a blood pressure measuring device (properly abbreviated as an electronic sphygmomanometer).

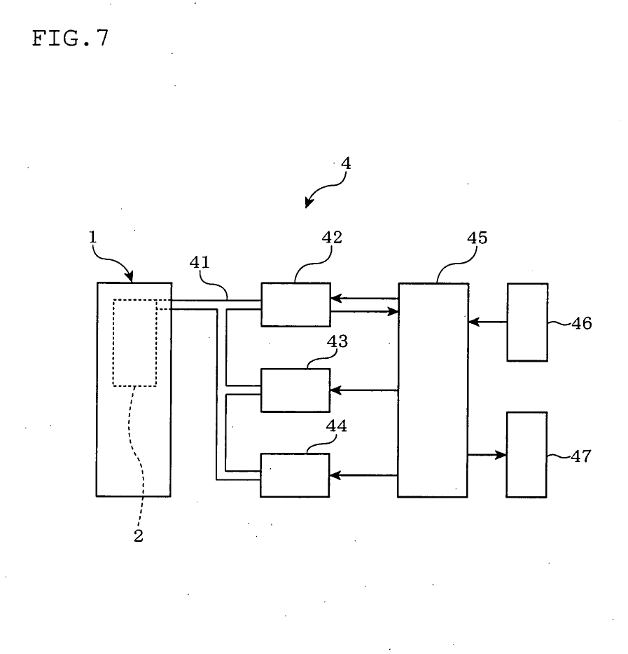

FIG. 7 shows a schematic block diagram of essential parts of an oscillometric electronic sphygmomanometer according to one embodiment of the present invention.

In FIG. 7, an oscillometric electronic sphygmomanometer 4 comprises the living body pressing device 1, a pressure sensor 42, a pump 43, an exhaust valve 44, an information processing unit 45, an operation switch 46 and a display 47. The living body pressing device 1 has the air bag 2 therein. The pressure sensor 42, the pump 43 and the exhaust valve 44 are in communication with the air bag 2 via a pipe 41. The information processing unit 45 is connected to the pressure sensor 42, the pump 43 and the exhaust valve 44. The operation switch 46 and the display 47 are connected to the information processing unit 45.

Here, the oscillometric electronic sphygmomanometer 4 according to the present embodiment has a configuration comprising the living body pressing device 1 of the embodiment described above.

Consequently, the oscillometric electronic sphygmomanometer 4 according to the present embodiment can uniformly press the arm as described above, and enables effective pressing at both ends in the arm-axis direction as described above, and can therefore accurately measure the blood pressure. Moreover, the living body pressing device 1 can be reduced in size.

While the preferred embodiments of the living body pressing device, the method of manufacturing the same, and the blood pressure measuring device according to the present invention have been shown and described above, it should be understood that the living body pressing device, the method of manufacturing the same, and the blood pressure measuring device according to the present invention are not limited to the embodiments described above, and various modifications can be made within the scope of the invention.

For example, the reinforcing sheet 3 is not exclusively mounted to the sheet 22 via the hand-side fused section 31 and the shoulder-side fused section 32. The reinforcing sheet 3 may be mounted to the sheet 22, for example, via the hand-side fused section 31, the shoulder-side fused section 32 and a plurality of fused sections (not shown) parallel to the arm-axis direction between the hand-side fused section 31 and the shoulder-side fused section 32.

### INDUSTRIAL APPLICABILITY

As described above, the living body pressing device according to the present invention is not exclusively used as a sphygmomanometer cuff, and can also be effectively applied as, for example, a stanching living body pressing device which requires an accurate measurement of a pulse wave during pressurization.

1. A living body pressing device comprising a fluid bag which inflates or deflates as a fluid comes in or out and which is wound around a living body,
wherein a reinforcing sheet is provided on a side of the fluid bag that does not face the living body.

2. The living body pressing device according to claim 1, wherein the fluid bag is rectangular, and the both ends of the reinforcing sheet on the long sides of the fluid bag are secured.

3. The living body pressing device according to claim 1, wherein the reinforcing sheet is secured in a region smaller than the fluid bag.

4. The living body pressing device according to claim 1, wherein the fluid bag is formed by folding a single sheet.

5. The living body pressing device according to claim 1, wherein one of fused sections in a short-side direction, where both ends of the reinforcing sheet in the short-side direction and the upper surface of the fluid bag are fused, overlaps the fused section of the upper surface.

6. The living body pressing device according to claim 1, wherein a sheet core is interposed between the upper surface of the fluid bag and the reinforcing sheet.

7. A method of manufacturing a living body pressing device which is wound around a living body and which comprises a fluid bag that inflates or deflates as a fluid comes in or out and that is equipped with a reinforcing sheet, and a cover including the fluid bag therein, the method comprising the steps of:
to manufacture the fluid bag,
folding both ends of a single sheet parallel to a long-side direction;
fusing the folded both ends and at least one of the both ends of the reinforcing sheet in the short-side direction at the same time; and
fusing the both ends in the long-side direction, respectively.

8. A blood pressure measuring device which uses the living body pressing device according to claim 1.
